(19) United States
(12) Patent Application Publication
(10) Pub. No.: US 2023/0272710 A1
(43) Pub. Date: Aug. 31, 2023

(54) SOLIDS ANALYSIS OF DRILLING AND COMPLETION FLUIDS

(71) Applicant: Board of Regents, The University of Texas System, Austin, TX (US)

(72) Inventors: Eric VAN OORT, Austin, TX (US); Sercan GUL, Irving, TX (US)

(21) Appl. No.: 18/018,350

(22) PCT Filed: Jul. 29, 2021
(86) PCT No.: PCT/US2021/043670

§ 371 Date: Jan. 27, 2023

Related U.S. Application Data
(60) Provisional application No. 63/058,961, filed on Jul. 30, 2020.

(51) Int. Cl. E21B 49/00; G01N 23/223

(52) U.S. Cl. CPC E21B 49/005; G01N 23/223; G01N 2223/076; G01N 2223/616

(57) ABSTRACT

An apparatus for solids analysis of a wellbore fluid includes a pipe formed from radiolucent material, the pipe having a bore for conveying the wellbore fluid. The apparatus includes an excitation source for generating source x-rays. The apparatus includes a collimator for directing the source x-rays to the wellbore fluid within the bore of the pipe. The apparatus includes a detector for receiving fluorescent x-rays emitted by a first element of the wellbore fluid within the bore of the pipe. The apparatus includes a processor for determining a concentration of a first solid in the wellbore fluid based on counting fluorescent x-rays having energy levels corresponding to the first element.

### BACKGROUND OF THE DISCLOSURE

### Field of the Disclosure

The present disclosure generally relates to solids analysis of drilling and completion fluids. More particularly, embodiments of the present disclosure relate to a system for x-ray fluorescence elemental analysis of drilling and completion fluids conveyed through a flow loop.

### Description of the Related Art

Drilling and completion of oil or gas wells usually requires circulating fluids through the well for maintaining optimum drilling performance, hole cleaning, ECD management, preventing failures of surface and downhole equipment during drilling, exerting hydrostatic pressure, and maintaining wellbore stability.

The fluids commonly include various solids, such as low-gravity solids (LGS) and/or high-gravity solids (HGS), which can be derived from the formation or deliberately added. For example, the LGS may include sands, clays, and other minerals entrained in the fluids during drilling of downhole rock formations, and the HGS may include barite, hematite, and other high density minerals that are added to increase fluid density.

It is desirable to maintain the various solids at optimum levels within the fluids throughout drilling and completion operations. To that end, field-based solids analysis may be used to monitor solids content. If a discrepancy between target and actual solids content is detected, mitigation techniques may be employed, namely by diluting the fluids or solids control.

The conventional solids analysis techniques performed at a well site (e.g. using retort kit involving distillation and analysis on collected solids) are time consuming, inherently unsafe, difficult to automate, and inaccurate. The conventional techniques are limited to about 3 analyses per day, require heating fluids to above 900° F., use manual human analysis, and can suffer from leakage and residual fluid.

Therefore, there is a need for an improved field-based solids analysis of drilling and completion fluids.

### SUMMARY OF THE DISCLOSURE

In one embodiment, an apparatus for solids analysis of a wellbore fluid includes a pipe formed from radiolucent material, the pipe having a bore for conveying the wellbore fluid. The apparatus includes an excitation source for generating source x-rays. The apparatus includes a collimator for directing the source x-rays to the wellbore fluid within the bore of the pipe. The apparatus includes a detector for receiving fluorescent x-rays emitted by a first element of the wellbore fluid within the bore of the pipe. The apparatus includes a processor for determining a concentration of a first solid in the wellbore fluid based on counting fluorescent x-rays having energy levels corresponding to the first element.

In another embodiment, a system for solids analysis includes a mud pit containing a fluid. The system includes a flow loop for receiving the fluid from the mud pit and returning the fluid to the mud pit. The flow loop includes a pipe formed from radiolucent material, the pipe having a bore for conveying the fluid. The system includes an x-ray based elemental analysis instrument disposed adjacent to the pipe.

In one embodiment, a method for solids analysis of a wellbore fluid includes conveying the wellbore fluid through a bore of a pipe formed from radiolucent material. The method includes generating source x-rays outside the pipe. The method includes directing the source x-rays to the wellbore fluid within the bore of the pipe. The method includes detecting x-ray fluorescence (XRF) signal intensity for a first element in the wellbore fluid. The method includes determining a concentration of a first solid in the wellbore fluid based on the XRF signal intensity for the first element.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 is a schematic diagram of a system for solids analysis of a fluid at a well site according to one embodiment of the present disclosure.

FIG. 2 is an enlarged sectional view taken along section line 2-2′ of FIG. 1 showing a schematic diagram of a handheld x-ray fluorescence (XRF) analyzer disposed adjacent to a pipe according to one embodiment of the present disclosure.

FIG. 3 is a flowchart illustrating a method for calibrating a system for determining solids content of a fluid according to one embodiment of the present disclosure.

FIGS. 4A-4C illustrate exemplary calibration curves for bentonite, barite, and sand, respectively, according to one embodiment of the present disclosure.

FIG. 5 is a flowchart illustrating a method for determining solids content of a fluid according to one embodiment of the present disclosure.

FIG. 6 is a flowchart illustrating a method for mud logging, according to certain embodiments.

### DETAILED DESCRIPTION

The present disclosure generally relates to solids analysis of drilling and completion fluids. More particularly, embodiments of the present disclosure relate to a system for x-ray fluorescence elemental analysis of drilling and completion fluids conveyed through a flow loop. Embodiments of the present disclosure relate to an apparatus for solids analysis of a wellbore fluid including a pipe formed from a radiolucent material, the pipe having a bore for conveying the wellbore fluid; an excitation source for generating source x-rays; a collimator for directing the source x-rays to the wellbore fluid within the bore of the pipe; a detector for receiving fluorescent x-rays emitted by a first element of the wellbore fluid within the bore of the pipe; and a processor for determining a concentration of a first solid in the wellbore fluid based on counting fluorescent x-rays having energy levels corresponding to the first element.

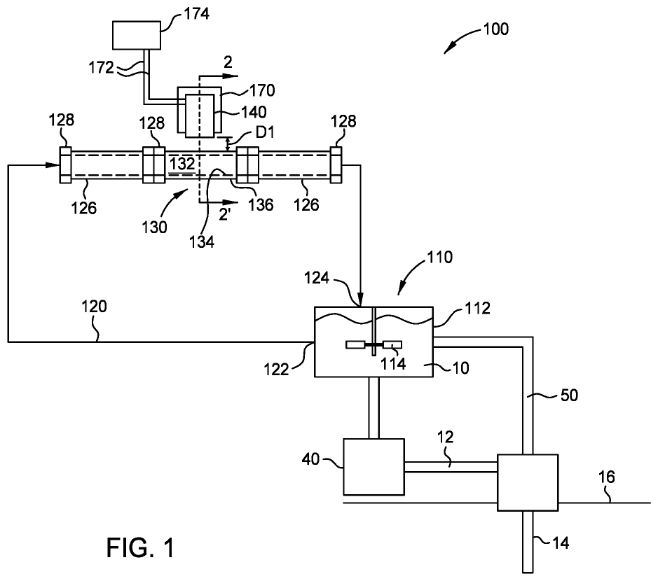

FIG. 1 is a schematic diagram of a system 100 for solids analysis of a fluid 10 at a well site according to one embodiment of the present disclosure. Referring to FIG. 1, the system 100 includes a mud pit 110, a flow loop 120, a pipe 130, and an x-ray fluorescence (XRF) analyzer 140.

In one embodiment, the fluid 10 can include various low-gravity solids (LGS). Exemplary LGS can be or include sands (e.g., silica, quartz, and other minerals consisting of silicon and oxygen, among others), clays (e.g., bentonite), and other minerals. In another embodiment, the fluid 10 can include various high-gravity solids (HGS). Exemplary HGS can be or include barite (barium sulfate), hematite (iron oxide), and other minerals. In one embodiment, the solids may be homogeneously dispersed in the fluid 10. In some other embodiments, cuttings and/or other heavy solids may be heterogeneously dispersed and suspended in the fluid 10. In some embodiments, the solids can be or include dissolved and/or undissolved solids.

In one embodiment, the fluid 10 can be or include a drilling fluid. In another embodiment, the fluid 10 can be or include a completion fluid. In some embodiments, the fluid 10 can be or include a return fluid 12 from a subterranean wellbore 14 formed in a rock formation 16. Drill cuttings carried by the return fluid 12 returning from the wellbore 14 may be removed in a shale shaker before the return fluid 12 reaches the mud pit 110. In some embodiments, the return fluid 12 may be circulated through one or more of a desander, a desilter, or a degasser (i.e., mud cleaning equipment 40) to remove contaminants from the return fluid 12, to condition the return fluid 12, or both.

In one embodiment, the fluid 10 can include fine solids which are characteristic of formation lithology. In such embodiments, apparatus and methods of the present disclosure may provide a lithological analysis of the formation 16. In some embodiments, apparatus and methods of the present disclosure may provide a petrophysical or mud logging analysis of the fluid 10.

The mud pit 110 may be in fluid communication with the mud cleaning equipment 40, e.g., the shale shaker. In one embodiment, the mud pit 110 may include one or more metal tanks 112 fitted with agitators 114 to maintain solids in suspension.

The mud pit 110 is also in fluid communication with the flow loop 120. The flow loop 120 includes an inlet 122 for receiving fluid 10 from the mud pit 110 and an outlet 124 for returning fluid 10 to the mud pit 110. In some embodiments, a pressure of the fluid 10 in the flow loop 120 may be from about 14.7 psi to about 500 psi, such as from about 100 psi to about 500 psi, such as from about 200 psi to about 300 psi. In some embodiments, a volumetric flow rate of the fluid 10 in the flow loop 120 may be from about 0.5 gal/min to about 20 gal/min. In some embodiments, a velocity of the fluid 10 may be from about 0.05 ft/s to about 33 ft/s.

As illustrated in FIG. 1, the flow loop 120 is off-line from the return fluid 12 returning from the wellbore 14 (i.e., wellbore return fluids). However, the flow loop 120 is not particularly limited to the illustrated embodiment. For example, the flow loop 120 may be in-line with the wellbore return fluids 12 such that one or more values of pressure, volumetric flow rate, or velocity for an in-line flow loop may exceed respective values for the off-line flow loop 120 described above. In some embodiments, the flow loop 120 may be in-line with a standpipe 50 of the well 20, such that the solids analysis is performed at high pressures relative to the off-line flow loop 120.

The flow loop 120 includes various pipe sections 126. In one embodiment, the pipe sections 126 may be formed of carbon steel or other materials having corrosion, chemical, temperature, and/or pressure resistance suitable for conveying the fluid 10. In one embodiment, the pipe sections 126 may have an inner diameter (ID) of from about 0.5 inches to about 2 inches. In some embodiments, the ID may be selected to avoid plugging. The pipe sections 126 are attached together at flanges 128 using suitable fasteners. In some other embodiments, the pipe sections 126 may be threaded together. In some embodiments, the flow loop 120 may include one or more pumps, valves, meters, and/or sensors.

The flow loop 120 also includes a pipe 130 formed from a radiolucent material and having a bore 132 for conveying the fluid 10. In certain embodiments, the flow loop 120 is omitted. Instead, the pipe 130 may be coupled in-line with existing piping. For example, the pipe 130 may be coupled between the wellbore 14 and the mud cleaning equipment 40. Alternatively, the pipe 130 may be coupled between the mud cleaning equipment 40 and the mud pit 110. Alternatively, the pipe 130 may be coupled on the standpipe 50. In one embodiment, the pipe 130 may be attached between first and second pipe sections 126 at respective flanges 128. The bore 132 of the pipe 130 is in fluid communication with the mud pit 110. In some embodiments, an ID of the pipe 130 may be from about 0.5 inches to about 2 inches. In some embodiments, the ID of the pipe 130 may match the ID of the pipe sections 126. In some embodiments, a thickness of the pipe 130, measured radially from an inner wall 134 to an outer wall 136, may be from about 0.05 inches to about 0.25 inches. In some embodiments, the thickness may be selected to have a burst pressure of about 50 psi or more, such as from about 50 psi to about 10,000 psi, such as from about 50 psi to about 7,500 psi, such as from about 50 psi to about 2,500 psi, alternatively from about 2,000 psi to about 5,000 psi, alternatively from about 5,000 psi to about 7,500 psi. In some embodiments, a length of the pipe 130 may be long enough to accommodate the XRF analyzer 140 between the flanges 128. In some embodiments, the length of the pipe 130 may be from about 1 inch to about 12 inches, such as from about 2 inches to about 6 inches. In some other embodiments, the length of the pipe 130 may be greater than 12 inches.

In certain embodiments, radiolucent materials include materials having an x-ray transparency of about 50% or more, such as about 60% or more, such as about 70% or more, such as about 80% or more, such as about 90% or more, such as about 95% or more, such as about 99% or more, such as about 99.9% or more. In some embodiments, radiolucent materials include materials having an x-ray transparency from about 50% to about 100%, such as from about 50% to about 60%, alternatively from about 60% to about 70%, alternatively from about 70% to about 80%, alternatively from about 80% to about 90%, alternatively from about 90% to about 95%, alternatively from about 95% to about 99%, alternatively from about 70% to about 99%, alternatively from about 99% to about 99.9%, alternatively from about 99.9% to about 100%, alternatively from about 90% to about 100%.

In some embodiments, the pipe 130 may be formed from one or more non-metal materials. In one embodiment, the pipe 130 may be formed from a polymeric, radiolucent material such as a polymer composite including one or more polymeric materials, one or more non-polymeric materials, or both. In another embodiment, the pipe 130 may be formed from carbon fiber. In one embodiment, the pipe 130 may be a carbon fiber composite or carbon fiber reinforced polymer. In yet another embodiment, the pipe 130 may be formed from polyethylene, polypropylene, or another suitable polymer.

In some embodiments, the pipe 130 may be formed of a material having an x-ray mass attenuation coefficient of about 7 cm²/g or less, such as about 2.5 cm²/g or less, such as about 1 cm²/g or less, such as about 0.5 cm²/g or less, such as about 0.25 cm²/g or less, such as about 0.1 cm²/g or less, such as about 0.01 cm²/g or less. In some embodiments, the pipe 130 may be formed of a material having an x-ray mass attenuation coefficient of from about 0.1 cm²/g to about 0.2 cm²/g, when exposed to x-ray energies of about 100 keV. In some embodiments, the pipe 130 may be formed of a material having an x-ray mass attenuation coefficient of from about 0.1 cm²/g to about 0.25 cm²/g, when exposed to x-ray energies of about 50 keV. In some embodiments, the pipe 130 may be formed of a material having an x-ray mass attenuation coefficient of from about 0.4 cm²/g to about 0.5 cm²/g, when exposed to x-ray energies of about 20 keV. In some embodiments, the pipe 130 may be formed of a material having an x-ray mass attenuation coefficient of from about 2 cm²/g to about 7 cm²/g, when exposed to x-ray energies of about 10 keV. In some embodiments, the pipe 130 may be formed of a material having an x-ray mass attenuation coefficient of from about 0.1 cm²/g to about 1 cm²/g, when exposed to x-ray energies of from about 20 keV to about 100 keV. In some embodiments, the pipe 130 may be formed of a material having an x-ray mass attenuation coefficient of from about 0.1 cm²/g to about 7 cm²/g, when exposed to x-ray energies of from about 10 keV to about 50 keV. In some embodiments, the pipe 130 may have an effective x-ray mass attenuation coefficient of from about 0.1 cm²/g to about 7 cm²/g, when used with a handheld XRF analyzer. In some embodiments, the pipe 130 may be substantially transparent to x-rays.

The system 100 also includes an XRF analyzer 140 disposed adjacent to the pipe 130. In some embodiments, a gap D1 between the XRF analyzer 140 and the outer wall 136 of the pipe 130 may be about 1 inch or less, such as about 0.75 inches or less, such as about 0.5 inches or less, such as about 0.25 inches or less, such as about 0.125 inches or less. In some embodiments, the XRF analyzer 140 may be in direct contact with the outer wall 136 of the pipe 130.

The system 100 may optionally include lead shielding 170 around the XRF analyzer 140. In some embodiments, cables 172 may be routed from an external data acquisition (DAQ) system 174 through the lead shielding 170 to the XRF analyzer 140. In some embodiments, the cables 172 may provide transmission of power, data, and/or other signals between the XRF analyzer 140 and the DAQ system 174. In some embodiments, the cables 172 can be or include power, USB, PCI, PXI, Ethernet, or combinations thereof.

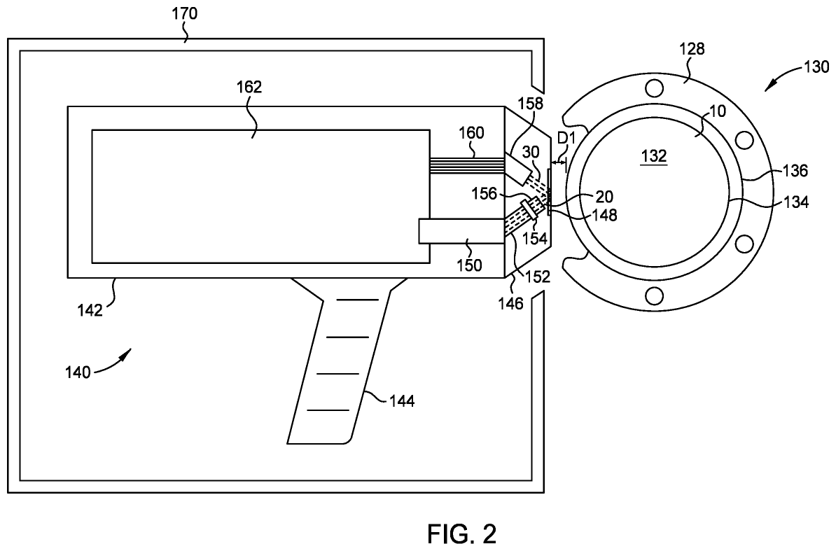

FIG. 2 is an enlarged sectional view taken along section line 2-2′ of FIG. 1 showing a schematic diagram of an XRF analyzer 140 disposed adjacent to a pipe 130 according to one embodiment of the present disclosure.

Referring to FIG. 2, the XRF analyzer 140 includes a housing 142 having an operator handle 144 attached to the housing 142. The XRF analyzer 140 also includes a nosepiece 146 attached to a front end of the housing 142. A front end of the nosepiece 146 includes a window 148 for preventing foreign material from entering the housing 142. The window 148 also provides electronic shielding and ambient light shielding while allowing x-rays to pass through. In some embodiments, the window 148 may be resistant to acids, bases, and organic chemicals. In some embodiments, the window 148 may be a thin film having a thickness of from about 5 μm or less. In one embodiment, the window 148 may be formed of a polymer substantially transparent to x-rays (e.g., prolene or ultralene). In some embodiments, the window 148 may be formed of polyethylene, e.g., ultra-high molecular weight polyethylene. In some embodiments, the window 148 may be gridded to add strength.

In one embodiment, the XRF analyzer 140 may be handheld. In some embodiments, the XRF analyzer 140 may be mounted to the pipe 130 by a clamp or another support frame (not shown). In some other embodiments, the XRF analyzer 140 may be a standalone instrument, a countertop instrument, or both.

The XRF analyzer 140 includes an excitation source 150 for generating source x-rays 20. The source x-rays 20 may also commonly be referred to as primary x-rays, and the terms may be used interchangeably herein. In some embodiments, the excitation source 150 may be an x-ray tube. In one embodiment, the excitation source 150 may be a Rhodium (Rh) thin window x-ray tube coupled to an x-ray generator operating at 6-50 kV and 4.5-195 μA current, maximum 4 Watt output. In some embodiments, the excitation source 150 may have operator adjustable current and voltage for optimum excitation. In some embodiments, different currents may result in different count rates.

The XRF analyzer 140 includes a collimator 152 for forming an x-ray beam consisting of parallel source x-rays 20. In some embodiments, the collimator 152 can be user changeable, e.g., including 3 mm and 8 mm collimators.

The XRF analyzer 140 may optionally include a filter 154 for filtering the source x-rays 20. In some embodiments, the filter 154 may be operator controlled, e.g., using a motorized filter wheel having multiple positions. In some embodiments, the housing 142 may include a slot for manual insertion filters (not shown).

The XRF analyzer 140 has a beam path 156 for directing the source x-rays 20 to the fluid 10 within the bore 132 of the pipe 130. In one embodiment, the beam path 156 may consist of a vacuum. In another embodiment, the beam path 156 may consist of helium. In yet another embodiment, the beam path 156 may consist of air. In one embodiment, the beam path 156 may be selectable from at least one of a vacuum, helium, and air.

In operation, the source x-rays 20 exit the nosepiece 146 through the window 148. The XRF analyzer 140 may be oriented such that the source x-rays 20 are directed toward the fluid 10. In some embodiments, the XRF analyzer 140 may be oriented such that the source x-rays 20 emitted through the window 148 are radially aligned with a centerline of the pipe 130 (i.e., intersecting a point along the centerline). In some embodiments, the XRF analyzer 140 may be oriented such that the source x-rays 20 emitted through the window 148 are directed along a transverse axis of the pipe 130 (i.e., in a transverse plane approximately perpendicular to a longitudinal axis of the pipe 130). In some embodiments, the XRF analyzer 140 may be oriented such that the source x-rays 20 emitted through the window 148 are within 45° of the transverse plane.

The source x-rays 20 may have an energy level greater than a binding energy of electrons in target atoms of the fluid 10. In some embodiments, the source x-rays 20 may be attenuated by passing through the pipe 130. Therefore, energy levels of the source x-rays 20 generated by the excitation source 150 and/or exiting the XRF analyzer 140 through the window 148 may be greater than an energy level of corresponding source x-rays 20 reaching the fluid 10. However, x-ray attenuation may be limited by using the pipe 130 of the present disclosure, such that source x-rays 20 may be generated using a handheld XRF analyzer 140 instead of requiring a high voltage x-ray generator.

In some embodiments, the source x-rays 20 may have an energy level of about 50 keV or less, such as from about 1 keV to about 50 keV, such as from about 1 keV to about 40 keV, such as from about 1 keV to about 30 keV, such as from about 1 keV to about 20 keV, such as from about 1 keV to about 10 keV, such as from about 1 keV to about 5 keV, alternatively from about 10 keV to about 50 keV, such as from about 5 keV to about 20 keV, such as from about 5 keV to about 10 keV. In some other embodiments, the source x-rays 20 may have an energy level of about 100 keV or less such as from about 1 keV to about 100 keV, such as from about 20 keV to about 100 keV.

The source x-rays 20 impacting the fluid 10 may cause x-ray fluorescence of the target atoms in the fluid 10 resulting in the emission of fluorescent x-rays 30. The fluorescent x-rays 30 may also commonly be referred to as secondary x-rays, and the terms may be used interchangeably herein.

In some embodiments, the fluorescent x-rays 30 may correspond to different spectral lines including Kα₁, Kβ₁, Lα₁, Lβ₁, and/or Mα₁. In one embodiment, a target element may be silicon, and the fluorescent x-rays 30 may have energies of about 1.74 keV (Kα₁) or 1.837 keV (Kβ₁). In another embodiment, the target element may be iron, and the fluorescent x-rays 30 may have energies of about 6.405 keV (Kα₁), 7.059 keV (Kβ₁), 0.705 keV (Lα₁), or 0.718 keV (Lβ₁). In yet another embodiment, the target element may be barium, and the fluorescent x-rays 30 may have energies of about 32.194 keV (Kα₁), 36.378 keV (Kβ₁), 4.466 keV (Lα₁), or 4.828 keV (Lβ₁). In yet another embodiment, the target element may be chlorine, and the fluorescent x-rays 30 may have energies of about 2.622 keV (Kα₁) or 2.812 keV (Kβ₁). In yet another embodiment, the target element may be sulfur, and the fluorescent x-rays 30 may have energies of about 2.309 keV (Kα₁) or 2.465 keV (Kβ₁). In yet another embodiment, the target element may be potassium, and the fluorescent x-rays 30 may have energies of about 3.314 keV (Kα₁) or 3.59 keV (pi). In yet another embodiment, the target element may be sodium, and the fluorescent x-rays 30 may have energies of about 1.04 keV (Kα₁). In yet another embodiment, the target element may be calcium, and the fluorescent x-rays 30 may have energies of about 3.692 keV (Kα₁), 4.013 keV (Kβ₁), 0.341 keV (Lα₁), or 0.345 keV (Lβ₁).

In some embodiments, XRF data may include a unique fluorescence signature, or array of fluorescence signatures, specific to one of a plurality of target elements and/or target solids. For example, a first type of quartz sand may have a first signature, and a second type of quartz sand may have a second signature, such that the first and second types of quartz sand may be distinguished from each other based on XRF data measured from the respective first and second types of quartz sand. One advantage of this approach is improved mud logging.

In some embodiments, the fluorescent x-rays 30 may have an energy level of from about 0.1 keV to about 40 keV, such as from about 0.3 keV to about 0.4 keV, alternatively from about 0.7 keV to about 0.8 keV, alternatively from about 1 keV to about 1.1 keV, alternatively from about 1.7 keV to about 1.8 keV, alternatively from about 1.8 keV to about 1.9 keV, alternatively from about 2.3 keV to about 2.4 keV, alternatively from about 2.4 keV to about 2.5 keV, alternatively from about 2.6 keV to about 2.7 keV, alternatively from about 2.8 keV to about 2.9 keV, alternatively from about 3.3 keV to about 3.4 keV, alternatively from about 3.5 keV to about 3.6 keV, alternatively from about 3.6 keV to about 3.7 keV, alternatively from about 4 keV to about 4.1 keV, alternatively from about 4.4 keV to about 4.5 keV, alternatively from about 4.8 keV to about 4.9 keV, alternatively from about 6.4 keV to about 6.5 keV, alternatively from about 7 keV to about 7.1 keV, alternatively from about 32.1 keV to about 32.2 keV, alternatively from about 36.3 keV to about 36.4 keV.

The fluorescent x-rays 30 enter the nosepiece 146 through the window 148. The XRF analyzer 140 also includes a detector 158 for receiving the fluorescent x-rays 30 emitted by target atoms of the fluid. In some embodiments, the detector 158 may be a silicon drift detector. In one embodiment, the detector 158 may include a detector window formed of beryllium or graphene. In one embodiment, the detector window may be an 8 μm beryllium window. In another embodiment, the detector window may be a 1 μm graphene window. In yet another embodiment, the detector window may be omitted (e.g., when operating in a helium atmosphere). In one embodiment, the detector 158 may have an active area of from about 20 mm² to about 30 mm². In some embodiments, the detector 158 may have a count rate of from about 40 kcps to about 250 kcps. In some embodiments, the detector 158 may have a lower resolution of about 140 eV or less at 250 kcps. In some embodiments, the detector 158 may detect fluorescent x-rays 30 having energy levels corresponding to atoms ranging from sodium (Na) to uranium (U). In some other embodiments, the detector 158 may detect fluorescent x-rays 30 having energy levels corresponding to atoms ranging from magnesium (Mg) to U.

The XRF analyzer 140 has electrical contacts 160 for transmitting power, signals, or both. The XRF analyzer 140 includes a processor 162 for receiving signals from the detector 158. The processor 162 may determine concentrations of elements present in the fluid 10 based on counting fluorescent x-rays 30 having energy levels corresponding to the respective elements. In some embodiments, the processor 162 can be or include one or more of an integrated circuit (IC), a printed circuit board (PCB), a programmable logic controller (PLC), a computer, or combinations thereof. The processor 162 may include memory.

In some embodiments, the XRF analyzer 140 can also include internal and/or external storage and wireless connectivity, e.g., via Wi-Fi or Bluetooth. In some embodiments, the XRF analyzer 140 may optionally include one or more of a Li-Ion battery, an internal camera, a touchscreen interface, an LCD display, input controls, a trigger switch, or combinations thereof. In some embodiments, the XRF analyzer 140 may be operated at temperatures of from about −10° C. to about 50° C.

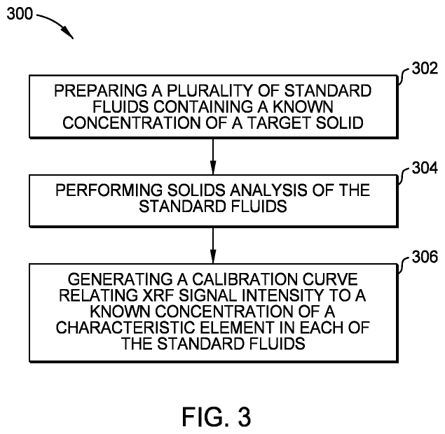

FIG. 3 is a flowchart illustrating a method 300 for calibrating a system for determining solids content of a fluid 10 according to one embodiment of the present disclosure. At block 302, the method 300 optionally includes preparing a plurality of standard fluids containing a known concentration of a target solid. In some embodiments, the number of standard fluids may be 3 or more, such as 4 or more, such as 5 or more, such as from 5 to 10, such as from 5 to 7, alternatively from 3 to 7. In some embodiments, the target solid may include sands, clays, salts, other minerals, other LGS, other HGS, other solids common to drilling and/or completion fluids, or combinations thereof. In some embodiments, the target solid can be one or more of barite, bentonite, hematite, sand, chlorides (e.g., potassium chloride, sodium chloride, calcium chloride lithium chloride, magnesium chloride), calcium carbonate, other clays (e.g., illite, smectite, kaolinite, chlorite, mica), hydroxides (e.g., potassium hydroxide, sodium hydroxide), formates (e.g., sodium formate, potassium formate, cesium formate), bromides (e.g., calcium bromide, zinc bromide), nitrates (e.g., sodium nitrate, potassium nitrate, magnesium nitrate, calcium nitrate, zinc nitrate), silicates (e.g., lithium silicate, sodium silicate, potassium silicate), manganese tetroxide, other iron oxides, ilmenite, galena, lignosulfonates, lignites, hydrocarbon-based compounds (alcohols, esters, ethers, olefins, paraffins, polymers), cellulose compounds, amines and ammonium salts, sulfonated asphalts, gilsonites, soda ash, lime, rock minerals (e.g., quartz, feldspars, plagioclase, pyrite, apatite, calcite, siderite, dolomite, hornblende, marcasite, basanite, heulandite, analcime, gypsum, halite, anhydrite, polyhalite, carnallite, langbeinite, kainite, kieserite, blödite, borax, epsomite, gaylussite, glauberite, mirabilite, thenardite, trona, granite, diorite, gabbro, peridodite, rhyolite, andesite, basalt, komatiite, coal), other solids, other materials that can be used as drilling fluid additives, other solids that are potentially identifiable by unique XRF signature(s), and combinations thereof. In some embodiments, the standard fluids can include a base fluid including at least one of oil-based drilling muds, water-based drilling muds, polymer muds (e.g., KCI polymer mud), other drilling muds, completion fluids, treatment fluids, cementing fluids, other wellbore fluids, or combinations thereof. In some embodiments, concentrations of the target solid may be measured on a volume or weight basis. In one embodiment, the standard fluids may include concentrations of the target solid in the base fluid of about 20% v/v or less, such as from about 0% v/v to about 20% v/v, such as from about 0% v/v to about 10% v/v.

At block 304, the method 300 optionally includes performing solids analysis of the standard fluids. In one embodiment, solids analysis of the standard fluids may be performed using the system 100 at a well site (i.e., field-based analysis), such as by using the XRF analyzer 140. In another embodiment, solids analysis of the standard fluids may be performed using a scaled-up version of the system 100 which is developed fit-for-purpose, e.g., for field deployment. In yet another embodiment, solids analysis of the standard fluids may be performed using a scaled-down and/or lab-based system physically and/or operationally equivalent to the system 100. In yet another embodiment, solids analysis of the standard fluids may be performed using a scaled-down and/or lab-based system physically and/or operationally similar and/or analogous to the system 100.

In some embodiments, performing the solids analysis may include running each of the standard fluids through the system 100 in sequential order. In some embodiments, the system 100 may direct source x-rays 20 to the standard fluid and receive fluorescent x-rays 30 emitted by x-ray fluorescence of various elements in the standard fluid. In some embodiments, the system 100 may record a count of fluorescent x-rays (i.e., photon counts) having energy levels corresponding to one or more characteristic elements of each of the target solids. In some embodiments, based on the photon counts, the system 100 may record XRF signal intensities of the one or more characteristic elements, where each XRF signal intensity equals a total photon count for the respective element. For example, when the target solid is barite, the system 100 may record XRF signal intensity for barium, sulfur, or both. In another example, when the target solid is one of bentonite or hematite, the system 100 may record XRF signal intensity for iron. In yet another example, when the target solid is sand, the system 100 may record XRF signal intensity for silicon. In yet another example, when the target solid is a salt including for example potassium chloride, sodium chloride, or calcium chloride, the system 100 may record XRF signal intensity for chlorine, potassium, sodium, and/or calcium.

At block 306, the method 300 includes generating a calibration curve relating XRF signal intensity to a known concentration of a characteristic element in each of the standard fluids. In some embodiments, the calibration curve may be based on one of v/v, w/w, or w/v of the target solid relative to the base fluid. In some embodiments, the calibration curve may be generated programmatically, automatically, or both. In some embodiments, the calibration curve may be generated via an on-board processor, e.g., processor 162. In some other embodiments, the XRF data may be transmitted to an external DAQ system 174 and the calibration curve may be generated at a processor associated with the DAQ system 174 or by another processor at a remote location.

In some embodiments, the calibration curve may be generated based on historical data, previous calibration data, solids analysis data, or any other pertinent data which is already available. In such embodiments, block 302 and block 304 may optionally be omitted from the method 300.

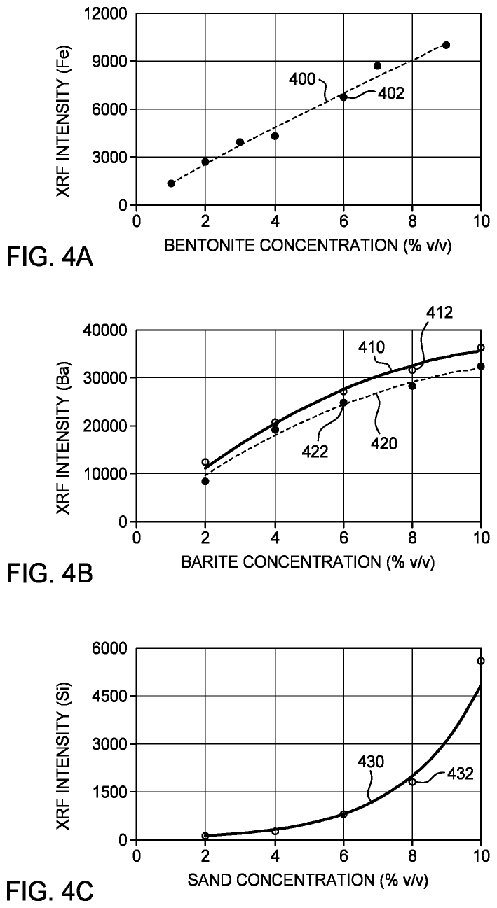

FIGS. 4A-4C illustrate exemplary calibration curves for bentonite 400, barite 410, 420, and sand 430, respectively, according to one embodiment of the present disclosure. In some embodiments, the calibration curves may independently fit at least one of a power law model, an exponential model, a polynomial model, a linear model, or another suitable statistical model. Referring to FIG. 4A, the calibration curve for bentonite 400 is fit to data points 402 representing XRF signal intensity for iron (i.e., a characteristic element of bentonite) measured from standard fluids including known bentonite concentrations (e.g., using the method 300). Referring to FIG. 4B, the calibration curve 410 is fit to data points 412, and the calibration curve 420 is fit to data points 422, each representing XRF signal intensity for barium (i.e., a characteristic element of barite) from two independent tests measured from standard fluids including known barite concentrations (e.g., using the method 300). Referring to FIG. 4C, the calibration curve 430 is fit to data points 432 representing XRF signal intensity for silicon (i.e., a characteristic element of sand) measured from standard fluids including known sand concentrations (e.g., using the method 300).

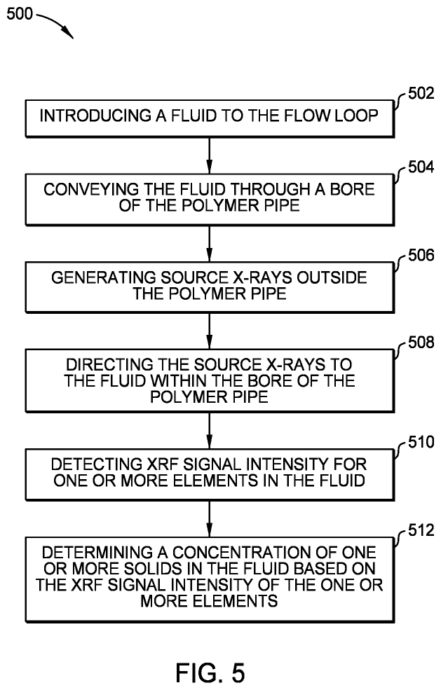

FIG. 5 is a flowchart illustrating a method 500 for determining solids content of a fluid 10 according to one embodiment of the present disclosure. In one embodiment, the method 500 may determine solids content of the fluid 10 using the system 100 of FIGS. 1-2. At block 502, the method 500 includes introducing the fluid 10 to the flow loop 120. The fluid 10 may enter the flow loop 120 from the mud pit 110 via the inlet 122.

At block 504, the method 500 includes conveying the fluid 10 through a bore 132 of the pipe 130.

At block 506, the method 500 includes generating source x-rays 20 outside the pipe 130. In some embodiments, the source x-rays 20 may be generated by the excitation source 150 of the XRF analyzer 140.

At block 508, the method 500 includes directing the source x-rays 20 to the fluid 10 within the bore 132 of the pipe 130. In some embodiments, the source x-rays 20 may be formed into an x-ray beam by the collimator 152, where the x-ray beam consists of parallel source x-rays 20. In some embodiments, the source x-rays 20 may be filtered through the optional filter 154. In some embodiments, the source x-rays 20 may be directed along the beam path 156. In some embodiments, the source x-rays 20 may exit the nosepiece 146 through the window 148.

The XRF analyzer 140 may be oriented such that the source x-rays 20 are directed toward the fluid 10. The source x-rays 20 impacting the fluid 10 may displace electrons from inner orbital shells of target atoms creating electron vacancies. The vacancies may be filled by higher orbit electrons moving down to the lower orbital shells, a process referred to as fluorescence. Higher orbit electrons have greater binding energies than lower orbit electrons, so as electrons move down, energy is emitted from the target atoms in the form of fluorescent x-rays 30. The amount of energy emitted is related to the distance between electron orbital shells. Since the distance between electron orbital shells is unique to each atom, the types and relative concentrations of target atoms present in the fluid 10 can be determined based on the energy levels of the fluorescent x-rays 30.

At block 510, the method 500 includes detecting XRF signal intensity for one or more elements in the fluid 10, such as by detecting the fluorescent x-rays 30 emitted by the target atoms in the fluid 10. The fluorescent x-rays 30 pass through the window 148 before reaching the detector 158. In some embodiments, the detector 158 may be substantially aligned with the source x-rays 20 exiting the window 148 and/or impacting the fluid 10 in order to increase an incidence of the fluorescent x-rays 30 upon the detector 158. In some embodiments, the detector 158 may be aligned within about 20° of the source x-rays 20, such as within about 10° of the source x-rays 20, such as within about 5° of the source x-rays 20.

In one embodiment, the processor 162 may determine a count of the fluorescent x-rays 30 having energy levels corresponding to characteristic elements of one or more target solids in the fluid 10. In other words, the processor 162 may determine XRF signal intensity for elements corresponding to one or more target solids in the fluid 10. In some embodiments, the target solid may include sands, clays, salts, other minerals, other LGS, other HGS, other solids common to drilling and/or completion fluids, or combinations thereof. In some embodiments, the target solid can be barite, bentonite, hematite, sand, potassium chloride, sodium chloride, calcium chloride, or another target solid described herein. For example, when the target solid is barite, the system 100 may record XRF signal intensity for barium. In another example, when the target solid is one of bentonite or hematite, the system 100 may record XRF signal intensity for iron, silicon, or aluminum. In yet another example, when the target solid is sand, the system 100 may record XRF signal intensity for silicon. In yet another example, when the target solid is one of potassium chloride, sodium chloride, calcium chloride, lithium chloride, or magnesium chloride, the system 100 may record XRF signal intensity for chlorine.

At block 512, the method 500 includes determining a concentration of one or more solids in the fluid 10 based on the XRF signal intensity of the one or more elements. In some embodiments, determining the concentration includes inputting the XRF signal intensity of a characteristic element corresponding to a first target solid to the calibration model for the first target solid. Inputting the XRF signal intensity to the calibration model results in calculating the concentration of the first target solid in the fluid 10.

In some embodiments, the XRF data may include a unique fluorescence signature, or array of fluorescence signatures, specific to one of a plurality of target elements and/or target solids. For example, a first type of quartz sand may have a first signature, and a second type of quartz sand may have a second signature, such that the first and second types of quartz sand may be distinguished from each other based on XRF data measured from the respective first and second types of quartz sand. In such embodiments, determining the concentration of the one or more solids in the fluid 10 may include determining relative concentrations of one or more types of the same solid.

In some embodiments, the concentration of the first target solid can be determined with error rates of about 0.2% or less, based on volumetric concentrations. In one embodiment, the method 500 can be or include a barite sag test. In some embodiments, the method 500 may use machine learning or other repeatable computational methods. In some other embodiments, qualitative information about the one or more elements may be determined. In some embodiments, data analysis may be performed using dimensionless parameters.

In some embodiments, the concentration of the first target solid may be determined programmatically, automatically, or both. In some embodiments, the concentration may be generated via an on-board processor, e.g., processor 162. In some other embodiments, the XRF data may be transmitted to an external DAQ system 174 and the concentration may be generated at a processor or PLC associated with the DAQ system 174 or by another processor at a remote location.

In some embodiments, the solids content of the fluid 10 may be determined in real-time, such as within about 1 min or less, such as within about 30 s or less, such as within about 10 s or less, such as within about 5 s or less, such as within about 1 s or less. In some embodiments, detection of elements having a concentration near a lower detection limit of the XRF analyzer 140 may require increased detection time. In some embodiments, the solids content of the fluid 10 may be determined faster than conventional techniques performed at the well site (e.g. using retort kit involving distillation and analysis on collected solids). For example, the retort method requires at least 2 h for testing and an additional 1 h for cooling and cleaning the equipment.

In some embodiments, the solids content of the fluid 10 may be determined at an increased frequency relative to conventional techniques. For example, the conventional techniques are limited to about 3 analyses per day, some of which may only be a partial analysis. In some embodiments of the present disclosure, the measurement frequency may be increased by one or more orders of magnitude relative to the conventional techniques. In some embodiments, the measurement frequency may be increased 2× or more, such as 5× or more, such as 10× or more, such as 100× or more. In some embodiments, the solids analysis may be performed at any desirable frequency, such as every 6 h or more, such as every 1 h or more, such as every 30 min or more, such as every 20 min or more, such as every 10 min or more, such as every 5 min or more, such as every 1 min or more, such as every 30 s or more, such as every 10 s or more. In other terms, the solids analysis may be performed more than 3× per day, such as 5× per day or more, such as 10× per day or more, such as 20× per day or more, such as 30× per day or more, such as 40× per day or more, such as 50× per day or more, such as 100× per day or more, such as 1,000× per day or more, such as 5,000× per day or more.

By determining solids content quickly and/or at relatively higher frequency compared to conventional techniques, various solids can be maintained closer to optimum levels within the fluid 10 throughout drilling and completion operations. In addition, if a discrepancy between target and actual solids content is detected, mitigation techniques may be employed in real-time, namely by diluting the fluid 10 or by performing solids control (e.g., solids separation and/or addition of chemicals). In some embodiments, the mitigation techniques can be employed within about 1 h or less, such as within about 30 min or less, such as within about 10 min or less, such as within about 5 min or less. Performing mitigation in real-time may limit and/or prevent solids build-up in the wellbore 14 that can increase abrasive wear, increase pump wear, reduce drilling speed, contribute to chip hold down effect, and/or detrimentally impact viscosity control.

In some embodiments, measuring the solids content of the fluid 10 in real-time, on-demand, or both can enable trend analysis of the data to be performed, such that predictions of mitigation action can be made in advance and deviations in solids content can be corrected more quickly.

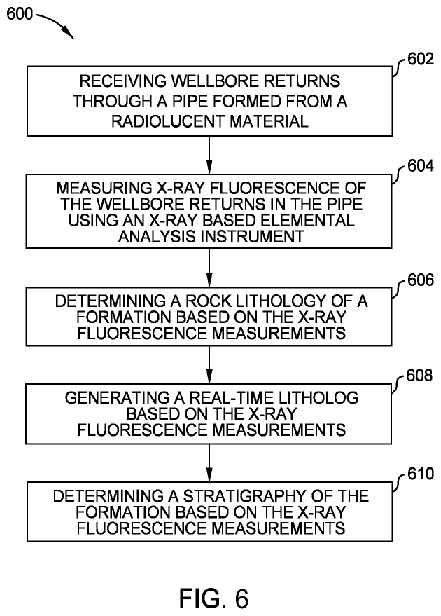

FIG. 6 is a flowchart illustrating a method 600 for mud logging. The method 600 may be implemented using the system 100 illustrated in FIGS. 1 and 2. At block 602, wellbore returns are received through a pipe formed from radiolucent material. The wellbore returns may include fine solids suspended in a drilling mud. The term “fine solids” may refer to colloidal solids of about 50 μm or smaller, such as about 5 μm or smaller. Fine solids remain in the mud even after the mud passes through the shale shaker. Embodiments of the present disclosure enable mud logging based on fine solids, whereas conventional mud loggers rely on larger drilling cuttings of about 1 mm or larger, such as about 1 cm or larger, which are sampled from the shale shaker.

At block 604, x-ray fluorescence of the wellbore returns in the pipe is measured using an x-ray based elemental analysis instrument. The x-ray fluorescence measurements may be performed in-line. The x-ray fluorescence measurements may include a plurality of x-ray fluorescence signatures corresponding to different rock lithologies. The x-ray fluorescence signatures may include multiple signatures of different elements. For example, x-ray fluorescence signatures for iron and aluminum may be analyzed, and ratios of the two elements compared to identify a type of clay lithology. In addition, x-ray fluorescence of the wellbore returns may be analyzed at a single frequency or at a range or combination of frequencies to analyze multiple x-ray fluorescence signatures at once.

At block 606, a rock lithology of a formation is determined based on the x-ray fluorescence measurements. As described above, the wellbore returns may include fine solids suspended in a drilling mud. The drilling mud also includes base solids that may interfere with accurate determination of rock lithology based on the x-ray fluorescence measurements. Therefore, baseline x-ray fluorescence of the drilling mud may be measured so that the x-ray fluorescence of the wellbore returns may be compared to the baseline, and thus, the determination of rock lithology may be based on the comparison. In certain embodiments, machine learning may be implemented to improve baseline measurements and/or improve the comparison between datasets including the detection of changes in x-ray fluorescence signatures.

At block 608, a real-time litholog is generated based on the x-ray fluorescence measurements. The term “litholog” may refer to a composite of lithological logging data of a formation. At block 610, a stratigraphy of the formation is determined based on the x-ray fluorescence measurements. The term “stratigraphy” may refer to a geologic sequence of different lithologies within a formation.

The solids analysis of the present disclosure can be performed directly on the fluid 10 contained within the pipe 130 without sampling or diversion and without using high temperatures and/or pressures. Thus, the solids analysis can be performed safely.

The solids analysis of the present disclosure can be performed directly on the fluid 10 being conveyed (i.e., flowing) through the pipe 130 without requiring extraneous mixing and without settling of the solids in the fluid 130. Thus, the solids analysis can be performed as the solids are substantially homogeneously dispersed in the fluid 10. In some embodiments, the fluid 10 may be flowing continuously through the flow loop 120 to enable on-demand measurement.

Aspects of the present disclosure can provide quantitative and/or qualitative solids analysis of any Newtonian and non-Newtonian fluid (e.g., process fluids used in the food industry, mining, cementing, and tunneling, among others).

The present disclosure describes apparatus and methods for elemental analysis using an XRF analyzer. However, the elemental analysis is not particularly limited to the illustrated embodiment. For example, in some other embodiments, the elemental analysis may be performed using x-ray absorption spectroscopy (XAS), other suitable x-ray based elemental analysis techniques, suitable x-ray based elemental analysis instruments configured to perform one or more of the elemental analysis techniques described herein, or combinations thereof.

In one or more embodiments described herein, an apparatus is disclosed for performing solids analysis of a wellbore fluid.

In one or more embodiments, the apparatus comprises a pipe formed from radiolucent material, the pipe having a bore for conveying the wellbore fluid.

In one or more embodiments, the apparatus comprises an excitation source for generating source x-rays.

In one or more embodiments, the apparatus comprises a collimator for directing the source x-rays to the wellbore fluid within the bore of the pipe.

In one or more embodiments, the apparatus comprises a detector for receiving fluorescent x-rays emitted by a first element of the wellbore fluid within the bore of the pipe.

In one or more embodiments, the apparatus comprises a processor for determining a concentration of a first solid in the wellbore fluid based on counting fluorescent x-rays having energy levels corresponding to the first element.

In one or more embodiments, the apparatus comprises an x-ray fluorescence (XRF) analyzer including each of the excitation source, the collimator, the detector, and the processor.

In one or more embodiments, the XRF analyzer is handheld.

In one or more embodiments, the XRF analyzer comprises a housing having a window, the source x-rays exit the housing through the window, and the fluorescent x-rays enter the housing through the window.

In one or more embodiments, a gap between the window and an outer wall the pipe is about 1 inch or less.

In one or more embodiments, the pipe is formed from a polymer material.

In one or more embodiments, the pipe is formed from a non-metal material.

In one or more embodiments, the pipe has an inner diameter of from about 0.5 inches to about 2 inches.

In one or more embodiments, the pipe has an x-ray mass attenuation coefficient of from about 0.1 cm²/g to about 7 cm²/g, when exposed to x-rays of from about 10 keV to about 50 keV.

In one or more embodiments, the bore of the pipe is in fluid communication with a mud pit.

In one or more embodiments, the wellbore fluid comprises at least one of low-gravity solids, high-gravity solids, or a combination thereof.

In one or more embodiments, the wellbore fluid within the bore of the pipe comprises at least one of a drilling fluid, a completion fluid, or a combination thereof.

In one or more embodiments, the first element is selected from the group consisting of: barium, iron, aluminum, silicon, and chlorine.

In one or more embodiments described herein, a system is disclosed for performing solids analysis.

In one or more embodiments, the system comprises a mud pit containing a fluid.

In one or more embodiments, the system comprises a flow loop for receiving the fluid from the mud pit and returning the fluid to the mud pit.

In one or more embodiments, the flow loop comprises a pipe formed from radiolucent material, the pipe having a bore for conveying the fluid.

In one or more embodiments, the system comprises an x-ray based elemental analysis instrument disposed adjacent to the pipe.

In one or more embodiments, the pipe is formed from at least one of a polymer material or a non-metal material.

In one or more embodiments, the x-ray based elemental analysis instrument is an x-ray fluorescence (XRF) analyzer.

In one or more embodiments, the XRF analyzer is oriented for directing source x-rays to the fluid within the bore of the pipe and for receiving fluorescent x-rays emitted by elements of the fluid within the bore of the pipe.

In one or more embodiments, the x-ray based elemental analysis instrument is an x-ray absorption spectroscopy (XAS) instrument.

In one or more embodiments, the flow loop further comprises steel pipe.

In one or more embodiments, a pressure of the fluid in the flow loop is from about 100 psi to about 500 psi.

In one or more embodiments, the fluid in the mud pit comprises at least one of a drilling fluid and a completion fluid returned from a subterranean well.

In one or more embodiments described herein, a method is disclosed for performing solids analysis of a wellbore fluid.

In one or more embodiments, the method comprises conveying the wellbore fluid through a bore of a pipe formed from radiolucent material.

In one or more embodiments, the method comprises generating source x-rays outside the pipe.

In one or more embodiments, the method comprises directing the source x-rays to the wellbore fluid within the bore of the pipe.

In one or more embodiments, the method comprises detecting x-ray fluorescence (XRF) signal intensity for a first element in the wellbore fluid.

In one or more embodiments, the method comprises determining a concentration of a first solid in the wellbore fluid based on the XRF signal intensity for the first element.

In one or more embodiments, the method comprises delivering the wellbore fluid from a mud pit to an inlet of the pipe and returning the wellbore fluid from an outlet of the pipe to the mud pit.

In one or more embodiments, the concentration of the first solid is determined programmatically.

In one or more embodiments, the first element is silicon, and the first solid is sand.

In one or more embodiments, the first element is at least one of iron, silicon, or aluminum, and the first solid is bentonite.

In one or more embodiments, the first element is barium, and the first solid is barite.

In one or more embodiments, the first element is chlorine, and the first solid is at least one of potassium chloride, sodium chloride, calcium chloride, lithium chloride, or magnesium chloride.

While the foregoing is directed to embodiments of the present disclosure, other and further embodiments may be devised without departing from the basic scope thereof, and the scope of the present disclosure is determined by the claims that follow.

1. An apparatus for solids analysis of a wellbore fluid, comprising:
a pipe formed from radiolucent material, the pipe having a bore for conveying the wellbore fluid;
an excitation source for generating source x-rays;
a collimator for directing the source x-rays to the wellbore fluid within the bore of the pipe;
a detector for receiving fluorescent x-rays emitted by a first element of the wellbore fluid within the bore of the pipe; and
a processor for determining a concentration of a first solid in the wellbore fluid based on counting fluorescent x-rays having energy levels corresponding to the first element.

2. The apparatus of claim 1, comprising an x-ray fluorescence (XRF) analyzer including each of the excitation source, the collimator, the detector, and the processor, and wherein the XRF analyzer is handheld.

3. The apparatus of claim 2, wherein the XRF analyzer comprises a housing having a window, wherein the source x-rays exit the housing through the window, and wherein the fluorescent x-rays enter the housing through the window, and wherein a gap between the window and an outer wall the pipe is about 1 inch or less.

4. The apparatus of claim 1, wherein the pipe is formed from at least one of a polymer material or a non-metal material.

5. The apparatus of claim 1, wherein the pipe has an inner diameter of from about 0.5 inches to about 2 inches, and wherein the bore of the pipe is in fluid communication with a mud pit.

6. The apparatus of claim 1, wherein the pipe has an x-ray mass attenuation coefficient of from about 0.1 cm²/g to about 7 cm²/g, when exposed to x-rays of from about 10 keV to about 50 keV.

7. The apparatus of claim 1, wherein the wellbore fluid comprises at least one of low-gravity solids, high-gravity solids, a drilling fluid, a completion fluid, or a combination thereof.

8. The apparatus of claim 1, wherein the first element is selected from the group consisting of: barium, iron, aluminum, silicon, and chlorine.

9. A system for solids analysis, comprising:
a mud pit containing a fluid;
a flow loop for receiving the fluid from the mud pit and returning the fluid to the mud pit, wherein the flow loop includes a pipe formed from radiolucent material, the pipe having a bore for conveying the fluid; and
an x-ray based elemental analysis instrument disposed adjacent to the pipe.

10. The system of claim 9, wherein the pipe is formed from at least one of a polymer material or a non-metal material.

11. The system of claim 9, wherein the x-ray based elemental analysis instrument is an x-ray fluorescence (XRF) analyzer.

12. The system of claim 11, wherein the XRF analyzer is oriented for directing source x-rays to the fluid within the bore of the pipe and for receiving fluorescent x-rays emitted by elements of the fluid within the bore of the pipe.

13. The system of claim 9, wherein the x-ray based elemental analysis instrument is an x-ray absorption spectroscopy (XAS) instrument.

14. The system of claim 9, wherein the flow loop further comprises steel pipe, and wherein a pressure of the fluid in the flow loop is from about 100 psi to about 500 psi.

15. The system of claim 9, wherein the fluid in the mud pit comprises at least one of low-gravity solids, high-gravity solids, a drilling fluid, or a completion fluid returned from a subterranean well.

16. A method for solids analysis of a wellbore fluid, comprising:
conveying the wellbore fluid through a bore of a pipe formed from radiolucent material;
generating source x-rays outside the pipe;
directing the source x-rays to the wellbore fluid within the bore of the pipe;
detecting x-ray fluorescence (XRF) signal intensity for a first element in the wellbore fluid; and
determining a concentration of a first solid in the wellbore fluid based on the XRF signal intensity for the first element.

17. The method of claim 16, further comprising delivering the wellbore fluid from a mud pit to an inlet of the pipe and returning the wellbore fluid from an outlet of the pipe to the mud pit.

18. The method of claim 16, wherein the concentration of the first solid is determined programmatically.

19. The method of claim 16, wherein the first element is silicon, and wherein the first solid is sand.

20. The method of claim 16, wherein the first element is at least one of iron, silicon, or aluminum, and wherein the first solid is bentonite.

21.-28. (canceled)
